(19) United States
(12) Patent Application Publication
(10) Pub. No.: US 2023/0381322 A1
(43) Pub. Date: Nov. 30, 2023

(54) METHOD FOR PREPARING A DNA NETWORK WITH CONTROLLED CRYSTAL STRUCTURE AND A METHOD FOR INJECTING DRUGS USING THE DNA NETWORK

(71) Applicant: UIF (University Industry Foundation), Yonsei University, Seoul (KR)

(72) Inventors: Young Hoon ROH, Seoul (KR); Keon Wook NAM, Seoul (KR); In Seok CHOI, Seoul (KR)

(21) Appl. No.: 18/164,118

(22) Filed: Feb. 3, 2023

(30) Foreign Application Priority Data
Feb. 3, 2022 (KR) 10-2022-0013991

(51) Int. Cl. A61K 47/54; C12N 15/115; C12P 19/34; C12N 15/11; A61K 47/69; A61K 31/704; A61P 35/00

(52) U.S. Cl. CPC A61K 47/549; C12N 15/115; C12P 19/34; C12N 15/111; A61K 47/6949; A61K 31/704; A61P 35/00; C12N 2310/16; C12N 2310/531; C12N 2320/32; C12N 2320/52

(57) ABSTRACT

The present invention relates to a method for preparing a DNA network with a controlled crystal structure and a method for injecting drugs using the DNA network, and more specifically, the present invention relates to a method for controlling crystallinity in a DNA network by controlling the content of DTT relative to magnesium chloride in a process of synthesizing a functional DNA network by amplifying a circular DNA loaded with a functional base sequence through rolling circle amplification. Further, the present invention provides the possibility of applying pH-sensitive drug-controlled-release during injection by loading an anticancer drug into the DNA network prepared above.

### CROSS-REFERENCE TO RELATED APPLICATION

This application claims priority to and the benefit of Korean Patent Application No. 10-2022-0013991. filed on Feb. 3, 2022, the disclosure of which is incorporated herein by reference in its entirety.

### BACKGROUND

### 1. Field of the Invention

The present invention relates to a method for preparing a DNA network with a controlled crystal structure and a method for injecting drugs using the DNA network.

### 2. Discussion of Related Art

DNA, which has recently drawn attention as a gene therapy agent, reacts to various biomicroenvironments including pH, temperature, and biomaterials along with third-generation drugs, so that research is being actively conducted on functional base sequences that induce secondary structural conversion. Although drug-controlled-release systems in the form of nano/microparticles and hydrogels have been developed using such sensitive DNA constructs, effective drug delivery and therapeutic effects have not been achieved due to the low physiological stability and loading amount of DNA.

In order to overcome the above limitations of DNA, studies of constructing DNA micro/nanostructures such as branch-types, origami-types, and polymer-types using nucleic acid nanoengineering and applying them as drug delivery systems have been reported. Further, studies of enhancing physiological stability by the improvement of physical properties by forming organic and inorganic composites through physical and chemical bonding with functional inorganic materials such as carbon nanotubes, graphene oxide, gold nanoparticles, and silver nanoclusters and imparting functionality inherent to inorganic materials have been reported.

Rolling circle amplification (RCA) is a nucleic acid nanotechnology that continuously replicates functional base sequences using DNA polymerase, and can synthesize organic and inorganic composite structures through self-assembly of high-density functional polymeric DNA during the replication process and magnesium pyrophosphate (MgPPi) crystals produced during the synthesis process. Although it has been reported that the resulting composite structure exhibits solid properties and maintains its form in a liquid phase due to the nano-sized porosity and structural stability caused by inorganic crystals, and exhibits meta-properties that take on liquid properties in the air, changes caused by the regulation of crystallinity have not been reported. In addition, research on changing the inorganic crystal size and type of a synthesized composite structure has been reported, and changes in morphological and physicochemical properties depending on the size and bioimaging functionality imparted by the inorganic crystal type have been reported. However, since the connection between controllability and structural functionality of a DNA network has not been considered, there is a need for research on the correlation between the secondary structure formation efficiency of a DNA base sequence and the stability of a composite.

### SUMMARY OF THE INVENTION

An object of the present invention is to provide a method for preparing a DNA network in which the MgPPi crystallinity of an organic-inorganic complex is controlled by controlling the rolling circle amplification reaction.

Another object of the present invention is to provide a pharmaceutical use of a polymeric DNA network prepared by the above-described method.

To achieve the objects, the present invention provides a method for preparing a polymeric DNA network with controlled crystallinity, the method including:
hybridizing a first single-stranded circular DNA including a stimulus-sensitive moiety and a first hybridization site and a second single-stranded circular DNA including a target-attachment moiety and a second hybridization site; and
synthesizing a polymeric DNA network through rolling circle amplification (RCA) in a reaction solution including the hybridized circular DNA, DNA polymerase, magnesium chloride (MgCl₂) and dithiothreitol (DTT),
wherein the crystallinity of MgPPi crystals in the polymeric DNA network is controlled by controlling the molar (M) ratio of DTT to magnesium chloride (MgCl₂) from 0.1 to 50.

The present invention also provides a polymeric DNA network prepared by the method for preparing a polymeric DNA network with controlled crystallinity.

The present invention also provides a drug delivery system including the polymeric DNA network; and a pharmaceutically acceptable carrier.

The present invention also provides an anticancer composition including the polymeric DNA network; and an anticancer drug.

The present invention also provides a method for treating cancer, the method including administering a therapeutically effective amount of the anticancer composition to a subject in need thereof.

In the present invention, the porosity of DNA network, the rate of producing DNA and MgPPi crystals, the DNA distribution morphology, and the like may be controlled through a relatively simple process by controlling the content of a Mg chelating agent relative to MgCl₂ in an enzymatic reaction process during rolling circle amplification, and through this, drug loading, functionality and stability may be controlled.

Furthermore, the present invention has the effect of providing a DNA network in which the difficulty in controlling the physical properties of an existing rolling circle amplification-based DNA network is overcome not only by controlling the stiffness and elasticity of the network through the regulation of an enzymatic reaction, but also by controlling the physiological stability according to the crystallinity to design the degree of degradation, and stability is increased by improving physical properties.

Further, although a high-density base sequence which is replicated through existing rolling circle amplification has a disadvantage in that it is difficult to impart effective functionality and use it, the present invention may optimize base sequence-based functionality according to crystallinity when various functional base sequences are introduced along with the inherent function of inorganic crystals by controlling the ratio of the inorganic crystals and DNA, and may be used as a drug delivery system through the optimization.

### BRIEF DESCRIPTION OF THE DRAWINGS

The above and other objects, features and advantages of the present invention will become more apparent to those of ordinary skill in the art by describing in detail exemplary embodiments thereof with reference to the accompanying drawings, in which:

FIG. 1 shows the synthesis of crystallinity-controlled pDNets by manipulating chelator concentration during the enzymatic RCA process;
A) Synthesis of pDNets with two types of functional circular DNA templates, where hybridization sites were designed to construct physically entangled 3D DNA networks, resulting in tunable crystallinity, DNA localization patterns, dynamic DNA functionality, and structural stability. B) Scheme of mechanism underlying the formation of organic-inorganic composite structure during RCA and the major controlling steps during the synthesis of a DNA microsponge within the network. DTT interacts with Mg ions, which inhibits MgPPi formation, influencing the degree of crystallinity of the synthesized DNA microsponge;

FIG. 2 shows morphological and structural characterization of crystallinity-controlled pDNets;
(A) Digital images of pDNet. (B) Fluorescence images of SYBR-stained pDNets analyzed using fluorescence microscopy and CLSM. Scale bar: 20 μm. (C) SEM and (D) STEM images of the pDNets. Scale bar: 2 μm and 500 nm;

FIG. 3 shows elemental and kinetic analyses of pDNets;
(A) Representative STEM-based EDS mapping of C, N, and Mg of pDNets. Scale bar: 1 μm. (B) Relative atom ratios of C, N, O, Mg, and P with respect to C in pDNets based on the results of STEM-based EDS. (C) PXRD patterns of pDNets and (D) results of UV-vis spectrometry of pDNets analyzed at 520 nm during RCA. (E) Real-time analysis of fluorescence generated from intercalation of SYBR Green II during RCA. Measurements were taken at intervals of 5 min until saturation of the fluorescence intensity;

FIG. 4 shows elemental analysis of crystallinity-controlled pDNets using STEM-based EDS mapping. pDNet-H, -M, and -L denote high, medium, and low crystallinity, respectively. Scale bar: 1 μm;

FIG. 5 shows a stiffness comparison of pDNets. Young's moduli of pDNets were obtained at a frequency of 1 Hz;

FIG. 6 shows mechanical analyses of pDNets;
(A) Frequency sweep and (B) Strain sweep of pDNets evaluated at a frequency of 1 Hz. (C) Representative digital image of pDNets displaying highly elastic properties and injectability;

FIG. 7 shows the controlled release of anticancer therapeutics by pDNets;
(A) Schematic illustration of the sequence-guided pH-responsive drug release via formation of i-motif structure upon exposure to acidic environment. (B) Drug loading efficiency evaluated with Dox after 2 h and 48 h incubation, as indicated using shaded and dotted columns, respectively. The representative images of pDNets incubated after 2 h are shown in the inset. (C) pH-responsive release of Dox mediated by i-motif structure formation (pH 5.0, shaded column; pH 6.4, dotted column; pH 7.4, cross-hatched column; **p<0.01 and ***p<0.005). (D) Enzyme-responsive release of Dox evaluated using varying concentrations of DNase I concentration at 1 h of incubation;

FIG. 8 shows the drug loading and release of pDNets for localized drug delivery platform. Digital images of pDNets demonstrating loading efficiency and pH-responsive release of pDNet-H, -M, and -L. (high, medium, and low crystallinity, respectively);

FIG. 9 shows micromorphological analysis of Dox-loaded pDNets. SEM images of pDNets after Dox-intercalation for 24 h. Scale bar: 2 μm;

FIG. 10 shows the evaluation of crystallinity of the sequence-guided functionality of pDNets. The disease-targeted pH-responsive release profile of doxorubicin (Dox) at (A) pH 5.0, (B) 6.4, and (C) 7.4. Intercalated Dox was released upon the structural switch of the i-motif sequences. pDNet-H, -M, and -L denote high, medium, and low crystallinity, respectively;

FIG. 11 shows the cell adhesive functionality of pDNets;
(A) Scheme of cell-adhesive aptamer functionality of pDNets in terms of the nucleolin expression level of HeLa and NIH/3T3 cells. CLSM images of GelRed-labeled pDNets (red) (B) with aptamer and (C) scrambled sequences incubated with 2.5×10⁵ of nucleolin-positive Hela cells or nucleolin-negative NIH/3T3 cells stained with calcein AM (green). (D) ImageJ analysis of a HeLa cell attached to aptamer-functionalized pDNets. (E) Viability of HeLa cells treated with equal volumes of Dox-released solutions of pH 5.0, 6.4, and 7.4. Free Dox (4 μM) was used to represent the case where Dox was completely released;

FIG. 12 shows the enzymatic stability of pDNets. Digital images of pDNet-H, -M, and -L (high, medium, and low crystallinity, respectively), demonstrating structural stability of DNA network based on the DNase I concentration and enzymatic release of Dox after 8 h of drug loading;

FIG. 13 shows the evaluation of aptamer-functionalized pDNets. CLSM images of GelRedlabeled pDNets incubated with cell media containing 2.5×105 (A) nucleolin-positive Hela or (B) nucleolin-negative NIH/3T3 cells for 1 h. The cell membranes were stained with calcein AM. Scale bar: 40 μm;

FIG. 14 shows the evaluation of the functionality of pDNets without a targeting moiety. CLSM images of GelRed-labeled pDNets synthesized with scrambled aptamer sequences and incubated with cell media containing 2.5×10⁵ of (A) nucleolin-positive Hela or (B) nucleolin-negative NIH/3T3 cells for 1 h. The cell membranes were stained with calcein AM. Scale bar: 40 μm;

FIG. 15 shows the evaluation of intracellular delivery of Dox via controlled release. pDNets were incubated at pH 6.4 for 32 h, and the Dox (red)-released incubation solutions were used to treat 1×105 HeLa cells for 2 h. The cell nuclei were stained with Hoechst (blue), and free Dox (final concentration, 40 μM) was used for treatment as a control, representing the complete release of the encapsulated drug. Scale bar: 20 μm;

FIG. 16 shows the evaluation of the applicability of pDNets for localized drug delivery;
(A) Average tumor volume mice (n=5) treated with single injection of PBS (control), free Dox, pDNet-H, pDNet-M, and pDNet-L during the treatment period of 8 days. (B) Tumor weight analyses of mice in each group on day 8. (C) Average body weight analyses of mice in each group during the treatment period. *p<0.05 and **p<0.01. (D) H&E staining of major organs (heart, kidneys, liver, lungs, and spleen) 8 days after the intratumoral administration of the crystallinity-controlled pDNets. Scale bar: 50 μm; and

FIG. 17 shows the evaluation of the in vivo therapeutic efficacy of pDNets. Ex vivo images of tumors from mice (n=5) treated with PBS (control), free Dox, pDNet-H, pDNet-M, and pDNet-L. Scale bar: 1 cm.

### DETAILED DESCRIPTION OF EXEMPLARY EMBODIMENTS

Hereinafter, the configuration of the present invention will be described in detail.

The present invention provides a method for preparing a polymeric DNA network with controlled crystallinity, the method including:
hybridizing a first single-stranded circular DNA including a stimulus-sensitive moiety and a first hybridization site and a second single-stranded circular DNA including a target-attachment moiety and a second hybridization site; and
synthesizing a polymeric DNA network through rolling circle amplification (RCA) in a reaction solution including the hybridized circular DNA, DNA polymerase, magnesium chloride (MgCl₂) and dithiothreitol (DTT),
wherein the crystallinity of MgPPi crystals in the polymeric DNA network is controlled by controlling the molar (M) ratio of DTT to magnesium chloride (MgCl₂) from 0.1 to 50.

The present invention also provides a polymeric DNA network prepared by the method for preparing a polymeric DNA network with controlled crystallinity.

The method of the present invention is characterized by being an one-pot process, in which rolling circle amplification is performed using two types of circular DNAs including circular DNA including a stimulus-sensitive moiety and circular DNA including a target-attachment moiety as templates, the rolling circle amplification is performed by controlling the molar (M) ratio of DTT to magnesium chloride (MgCl₂) from 0.1 to 50, and as the molar ratio decreases, the crystallinity of MgPPi crystals in a polymeric DNA network increases, thereby controlling the crystallinity of the MgPPi crystals in the polymeric DNA network.

By controlling the crystallinity of the MgPPi crystals in the polymeric DNA network, localization patterns may be manipulated, drug loading and release profiles may be varied through the modulation of nanoporosity, and an ultrasoft viscoelastic mechanical property, structural stability having biological stability under physiological conditions, and the like may be controlled.

Furthermore, as circular DNA is loaded with functional base sequences which can be attached to target cells, the DNA network may offer effective tumor target attachment and subsequent potential for in vitro and in vivo antitumor therapy.

The steps of the method for preparing a polymeric DNA network with controlled crystallinity according to the present invention are as follows.

The first step is a hybridization step of circular DNAs loaded with functional base sequences.

The circular DNA consists of a first single-stranded circular DNA including a stimulus-sensitive moiety and a first hybridization site and a second single-stranded circular DNA including a target-attachment moiety and a second hybridization site.

The first single-stranded circular DNA includes a stimulus-sensitive moiety and a first hybridization site capable of hybridizing with a second single-stranded circular DNA.

The stimuli-sensitive moiety may be, for example, a pH-sensitive I-motif, a biological material-sensitive aptamer, an ion-sensitive G-quadraplex, a heat-sensitive double helix structure, a base sequence-sensitive toe-hold structure, or a base sequence and an element-sensitive DNAzyme and the like.

Further, the stimulus-sensitive moiety forms a secondary structure (for example, a hairpin structure) in a functional DNA sequence, and may be used as a drug-loading region.

The second single-stranded circular DNA includes a target-attachment moiety and a second hybridization site capable of hybridizing with a first single-stranded circular DNA.

A target-specific drug can be delivered due to the target-attachment moiety.

The target-attachment moiety may be an aptamer specific for cell receptors, ATP, ions or metal particles, and the like.

The first single-stranded circular DNA and the second single-stranded circular DNA may further include a primer for expressing a functional base sequence. As the primer, a known primer such as a T7 primer may be appropriately adopted and used.

In addition, the first hybridization site and the second hybridization site participating in the hybridization of two circular DNAs may include 10 to 40, preferably 10 to 20 complementary nucleotides. More preferably, the first hybridization site and the second hybridization site may be nucleotides disclosed in Table 1.

Therefore, preferably, the first single-stranded circular DNA may include a base sequence of SEQ ID NO: 1 including a stimulus-sensitive moiety and a first hybridization site. The second single-stranded circular DNA may include a base sequence of SEQ ID NO: 2 including a target-attachment moiety and a second hybridization site.

In addition, after hybridizing the first single-stranded circular DNA and the second single-stranded circular DNA, they may be ligated using a ligase such as T4 ligase.

The second step is a step in which the hybridized circular DNA, DNA polymerase, magnesium chloride (MgCl₂), DTT and a reaction buffer are mixed, a polymeric DNA network is synthesized through rolling circle amplification, and in this case, the crystallinity of MgPPi crystals in the polymeric DNA network is controlled by controlling the molar (M) ratio of DTT to the magnesium chloride (MgCl₂) from 0.1 to 50.

The hybridized circular DNA may synthesize a polymeric DNA network through the rolling circle amplification after the ligation thereof. As the molar (M) ratio of DTT to magnesium chloride (MgCl₂) decreases, the crystallinity of the MgPPi crystals increases. The crystallinity of MgPPi crystals may control the release rate of a loaded drug and modulate therapeutic efficacy.

Therefore, the molar ratio of DTT to magnesium chloride (MgCl₂) may range from 0.1 to 50.

According to an exemplary embodiment of the present invention, the drug delivery application of a functional DNA network whose crystal structure is modulated was identified using a pH-sensitive drug-releasing base sequence as a stimulus-sensitive moiety. For this purpose, circular DNA, in which an I-motif that is a pH-sensitive base sequence, a double helix hairpin structure that can be loaded with a drug, and a complementary binding base sequence for network stability were introduced, was designed. As a result of performing rolling circle amplification and performing a surface and structural analysis by controlling the ratio of DTT to MgCl₂ to 0.5, 10, and 20 based on the circular DNA loaded with the functional base sequence, it was confirmed that crystallinity inversely proportional to the proportion of DTT was formed. The DNA networks were named pDNet-H, pDNet-M and pDNet-L (polymeric DNA networks) according to the crystallinity. It was confirmed that the transparency of the network decreased as the crystallinity of the DNA network increased, and it was confirmed that the distribution of DNA in pDNet-H was mainly concentrated as a flower-like microstructure. Conversely, in the case of pDNet-L, it was confirmed that a transparent network was formed and DNA was mainly distributed in the foam of a polymeric network, confirming that the distribution of DNA can be controlled through the regulation of the rolling circle amplification reaction. When a physicochemical analysis according to the conditions of each rolling circle amplification, it was confirmed that the DNA and crystal formation rates were higher in the order of pDNet-M, pDNet-L, and pDNet-H, indicating that the efficiency of DNA polymerase is optimized according to the DTT proportion. In addition, as a result of comparing the degree of hardness and toughness of each DNA network by evaluating the physical properties using a rheometer, it was confirmed that the hardness of the network varied up to 6-fold in proportion to the efficiency of DNA polymerase and the degree of toughness varied up to 3-fold in proportion to the crystal content. Furthermore, as the crystallinity was increased by loading an anticancer drug, doxorubicin (Dox) intercalated into the double helix structure of DNA, the drug loading amount increased a maximum of 2.5-fold, and it was observed that the lower the crystallinity, the higher the drug release amount when exposed to a physiological environment whose pH was 5.0 (in cells), 6.4 (cancer tissue), and 7.4 (normal tissue), so that it was found that the lower the crystal content, the more efficient the secondary structure formation of I-motif sequences. However, in the case of pDNet-H, a significant increase (about 9-fold) in physiological stability to a DNase I enzyme was observed compared to pDNet-L. Finally, as a result of confirming the anticancer drug of the DNA network at the animal level, it was confirmed that the lower the crystallinity, the higher the drug release, similarly to the above drug release behavior, and thus, a high cancer tumor inhibitory effect was shown, and it was confirmed through body weight and H&E staining of tissue that the drug is not toxic due to local treatment by injection administration.

Therefore, the present invention also provides a drug delivery system including the polymeric DNA network; and a pharmaceutically acceptable carrier.

The polymeric DNA network of the present invention includes a stimulus-sensitive moiety, and may be used as a drug delivery system because the stimulus-sensitive moiety can be loaded with a drug. Furthermore, as the crystallinity is controlled, the release rate and release amount of the drug can be controlled, so that therapeutic efficacy can be improved.

Further, the polymeric DNA network of the present invention includes a target-attachment moiety, and thus, may be used as a target-specific drug delivery system.

The drug may be, for example, a generic medicine, drug, or prodrug. Examples thereof include: cardiovascular drugs, particularly, antihypertensive drugs (for example, calcium channel blockers, or calcium antagonists) and antiarrhythmic agents; congestive heart failure drugs; muscle contractants; vasodilators; ACE inhibitors; diuretics; carbonic anhydrase inhibitors; cardiac glycosides; phosphodiesterase inhibitors; blockers; β blockers; sodium channel blockers; potassium channel blockers; β-adrenergic agonists; platelet aggregation inhibitors; angiotensin II antagonists; anticoagulant drugs; thrombolytic agents; bleeding therapeutic agents; antianemic agents; thrombin inhibitors; antiparasitic agents; antibacterial agents; anti-inflammatory drugs, particularly, non-steroidal anti-inflammatory drugs (NSAIDs), more particularly COX-2 inhibitors; steroidal anti-inflammatory drugs; preventative anti-inflammatory drugs; antiglaucoma agents; mast cell stabilizers; mydriatics; drugs that affect the respiratory system; allergic rhinitis drugs; alpha-adrenergic antagonists; corticosteroids; chronic obstructive pulmonary disease drugs; xanthine-oxidase inhibitors; antiarthritic agents; gout medications; potent drugs and potent drug antagonists; anti-mycobacterium tuberculosis agents; antifungal agents; antiprotozoal agents; parasiticides; antiviral agents, particularly, respiratory antiviral agents, and antiviral agents for herpes, cytomegalovirus, human immunodeficiency virus, and hepatitis infection; therapeutic agents for leukemia and Kaposi's sarcoma; pain management agents, particularly, opioids including anesthetics and analgesics, opioid receptor agonists, opioid receptor partial agonists, opioid antagonists, opioid receptor mixed agonist-antagonists; neuroleptics; sympathomimetic agents; adrenergic antagonists; drugs affecting neurotransmitter absorption and release; anticholinergic agents; antihemorrhagic agents; prophylactic or therapeutic agents for radiation or chemotherapy effects; adipogenic agents; hypolipidemic agents; antiobesity agents such as lipase inhibitors; sympathomimetic agents; therapeutic agents for gastric ulcers and inflammation such as proton pump inhibitors; prostaglandins; VEGF inhibitors; antihyperlipidemic agents, particularly, statins; drugs that affect the central nervous system (CNS), for example, antipsychotic, antiepileptic and antiseizure drugs (anticonvulsants), psychoactive drugs, stimulants, antianxiety and hypnotics; antidepressants; antiparkinson's pharmaceuticals; hormones such as sex hormones and fragments thereof; growth hormone antagonists; gonadotropin releasing hormones and analogues thereof; steroid hormones and antagonists thereof; selective estrogen modulators; growth factors; antidiabetic agents such as insulin, insulin fragments, insulin analogues, glucagon-like peptides and antihypoglycemic agents; H1 , H2, H3 and H4 antihistamines; peptides, proteins, polypeptides, nucleic acids and oligonucleotide drugs; analogues, fragments and variants such as natural proteins, polypeptides, oligonucleotides and nucleic acids; drugs used to treat migraine headaches; asthma pharmaceuticals; cholinergic antagonists; glucocorticoids; androgens; antiandrogens; inhibitors of adrenocorticoid biosynthesis; therapeutic agents for osteoporosis, such as biphosphonates; antithyroid agents; sunscreens, sun protectants and filters; cytokine antagonists; antitumor agents; anti-Alzheimer's agents; HMGCoA reductase inhibitors; fibrates; cholesterol absorption inhibitors; HDL cholesterol elevating agents; triglyceride reducing agents; anti-aging or anti-wrinkle agents; precursor molecules for the generation of hormones; proteins such as collagen and elastin; antibacterial agents; anti-acne agents; antioxidants; hair treatments and skin whitening agents; variants of human apolipoproteins; precursor molecules for the generation of hormones; proteins and peptides thereof amino acids; plant extracts such as grape seed extract; DHEA; isoflavones; nutritional agents including vitamins, phytosterols and iridoid gylcosides, sesquiterpene lactones, terpenes, phenolic glycosides, triterpenes, hydroquinone derivatives, phenylalkanones; antioxidants such as retinol and other retinoids including retinoic acid and co enzyme Q10; omega-3-fatty acids; glucosamine; nucleic acids, oligonucleotides, antisense pharmaceuticals; enzymes; coenzymes; cytokine analogues; cytokine agonists; cytokine antagonists; immunoglobulins; antibodies; antibody pharmaceuticals; gene therapies; lipoproteins; erythropoietin; vaccines; small molecule therapeutic agents for the treatment or prevention of human and animal diseases such as allergies/asthma, arthritis, cancer, diabetes, growth impairment, cardiovascular diseases, inflammation, immunological disorders, baldness, pain, ophthalmological diseases, epilepsy, gynecological disorders, CNS diseases, viral infections, bacterial infections, parasitic infections, GI diseases, obesity, and hematological diseases, and the like, but are not limited thereto.

The nucleic acid drug may have a form such as deoxyribonucleic acid (DNA), ribonucleic acid (RNA), a peptide nucleic acid (PNA), a morpholino and locked nucleic acid (LNA), a glycol nucleic acid (GNA), an oliogonucleotide, plasmid DNA, an antisense oligonucleotide, messenger RNA, microRNA, a locked nucleic acid, a DNAzyme small interfering RNA, short hairpin RNA, an RNA-based enzyme (RNAzyme) or a nucleic acid aptamer. The nucleic acid may also include a sequence encoding one or more proteins or a non-coding sequence.

As an example of the nucleic acid drug, a nucleic acid including a coding and/or non-coding sequence such as polo-like kinase 1 (PLK1), apoptotic B-cell lymphoma 2 (Bcl-2), a brain derived neurotrophic factor (BDNF), a glial derived neurotrophic factor (GDNF), neurotrophic factor 3 (NT3), a fibroblast growth factor (FGF), a transforming growth factor (TGF), a platelet-derived transforming growth factor (PDGF), a milk growth factor (MGF), an endothelial growth factor (EGF), an endothelial cell-derived growth factor (ECDGF), a nerve growth factor (NGF), a vascular endothelial growth factor (VEGF), a 4-1 BB receptor (4-1BBR), a TNF-related apoptosis inducing ligand (TRAIL), artemin (GFR alpha3-RET ligand), B cell-attracting chemokine 1 (CXCL13), a B lymphocyte chemoattractant (BLC), a B cell maturation protein (BCMA), a bone-derived growth factor (BDF), a megakaryocyte derived growth factor (MGDF), a keratinocyte growth factor (KGF, thrombopoietin), a platelet-derived growth factor (PGDF), a megakaryocyte derived growth factor (MGDF), a keratinocyte growth factor (KGF), bone morphogenetic protein 2 (BMP2), BRAK, C-10, or Cardiotrophin 1 (CT1) may be introduced in the above-described various forms applicable to the human body.

The present invention also provides an anticancer composition including the polymeric DNA network; and an anticancer drug.

The polymeric DNA network of the present invention includes a stimulus-sensitive moiety, and may be used to treat cancer because the stimulus-sensitive moiety can be loaded with a drug, that is, an anticancer drug. Furthermore, as crystallinity is controlled, the release rate and release amount of the drug can be controlled, so that anticancer therapeutic efficacy can be improved.

Further, since the polymeric DNA network of the present invention includes a target-attachment moiety, the polymeric DNA network of the present invention may be used as a drug delivery system to enhance anticancer effects when a cancer cell-specific functional sequence is used as the target-attachment moiety.

The anticancer drugs are a general term for drugs that act on various metabolic pathways of cancer cells to exhibit cytotoxic or growth-inhibitory effects (cytostatic effects) on cancer cells, and may be classified into metabolic antagonists, plant alkaloids, topoisomerase inhibitors, alkylating agents, anticancer antibiotics, hormones or other drugs.

The anticancer drug may be oxaliplatin, imatinib, docetaxel, pemetrexed, gefitinib, tegafur, capecitabine, erlotinib, doxifluridine, paclitaxel, Interferon-α, gemcitabine, fludarabine, irinotecan, carboplatin, cisplatin, taxotere, doxorubicin, epirubicin, 5-fluorouracil, UFT, tamoxifen, goserelin, herceptin, an anti-CD20 antibody, leuprolide (Lupron), flutamide or the like, but is not limited thereto.

The cancer may be colorectal cancer, gastric cancer, liver cancer, lung cancer, breast cancer, biliary tract cancer, gallbladder cancer, pancreatic cancer, cervical cancer, esophageal cancer, brain cancer, rectal cancer, prostate cancer, head and neck cancer, or the like.

A pharmaceutically acceptable carrier that can be used in the drug delivery system or pharmaceutical composition of the present invention includes a carrier and a vehicle typically used in the medical field, and specific examples thereof include an ion exchange resin, alumina, aluminum stearate, lecithin, a serum protein (for example, human serum albumin), a buffer substance (for example, various phosphates, glycine, sorbic acid, potassium sorbate, partial glyceride mixtures of saturated vegetable fatty acids), water, a salt or electrolyte (for example, protamine sulfate, dissodium hydrogen phosphate, potassium hydrogen phosphate, sodium chloride, and zinc salts), colloidal silica, magnesium trisilicate, polyvinylpyrrolidone, cellulose-based substrates, polyethylene glycol, sodium carboxymethylcellulose, polyarylate, wax, polyethylene glycol, wool, or the like, but are not limited thereto.

In addition, the drug delivery system or pharmaceutical composition of the present invention may additionally include a lubricant, a wetting agent, an emulsifier, a suspending agent, a preservative, or the like, in addition to the aforementioned ingredients.

The drug delivery system or pharmaceutical composition of the present invention may be administered orally, rectally, transdermally, intravenously, intramuscularly, intraperitoneally, intramedullary, intrathecally, intradermally, or the like.

Formulations for oral administration may be tablets, pills, soft or hard capsules, granules, powders, liquids or emulsions, but are not limited thereto. Formulations for parenteral administration may be injections, drops, lotions, ointments, gels, creams, suspensions, emulsions, suppositories, patches or sprays, but are not limited thereto.

The drug delivery system or pharmaceutical composition of the present invention may include a diluent, an excipient, a lubricant, a binder, a disintegrant, a buffer, a dispersant, a surfactant, a colorant, a flavorant or a sweetener, if necessary. The drug delivery system or pharmaceutical composition of the present invention may be prepared by a typical method in the art.

An active ingredient of the drug delivery system or pharmaceutical composition of the present invention may vary depending on the age, sex, body weight, pathological condition, and severity of a subject to be administered, administration route, or judgment of a prescriber. The determination of dosage based on these factors is within the level of those skilled in the art, and the daily dose thereof may be, for example, 1 ng/kg/day to 10 mg/kg/day, specifically 10 ng/kg/day to 1 mg/kg/day, more specifically 0.1 μg/kg/day to 100 μg/kg/day, and even more specifically 0.2 μg/kg/day to 20 μg/kg/day, but is not limited thereto. The composition of the present invention may be administered once to three times a day, but is not limited thereto.

The present invention also provides a method for treating cancer, the method including administering a therapeutically effective amount of the anticancer composition to a subject in need thereof.

The subject may be a human or an animal other than a human, for example, a non-human animal such as a cow, a monkey, a bird, a cat, a mouse, a rat, a hamster, a pig, a dog, a rabbit, a sheep, and a horse.

In the treatment method of the present invention, the formulation, administration method, and the like of the composition are as described above.

Hereinafter, the present invention will be described in more detail through the Examples according to the present invention, but the scope of the present invention is not limited by the Examples suggested below.

### Example 1 Synthesis of Crystallinity-Controlled Polymeric DNA Networks and Characterization

(Materials)

DNA oligonucleotides and Tris-EDTA (TE) buffer were purchased from Integrated DNA Technology (Coralville, IA, USA). T4 DNA ligase (cat. No. M1804) was obtained from Promega (Madison, WI, USA) and φ29 DNA polymerase (part No. P7020-LC-L) was obtained from Enzymatics Inc. (Beverly, MA, USA). The premixed dNTP solution was procured from Lucigen (Madison, WI, USA). DTT and DNase I were purchased from Sigma Aldrich (St. Louis, MO, USA) and KCl was purchased from Duksan Pure Chemicals (Ansan, South Korea).

(Synthesis of Crystallinity-Controlled Polymeric DNA Networks)

Circular DNA was synthesized using 1 μM phosphorylated single-stranded DNA encoding anti-sense i-motif sequences and a single-stranded DNA encoding T7 promoter sequences in TE buffer. These two DNA strands were mixed and hybridized by heating the solution for 30 s at 95° C. and gradually cooling down to 20° C. using a PCR thermal cycler (T100™ Thermal Cycler, Bio-Rad, Hercules, CA, USA). For ligation, the hybridized DNA was incubated with 0.06 U/μL T4 ligase and the ligation reaction buffer [300 mM Tris-HCl (pH 7.8), 100 mM MgCl₂, 10 mM ATP, 100 mM DTT] at 23° C. for 24 h. pDNets with different crystallinities were synthesized by incubating circular DNAs at the final concentration of 0.3 μM with 5, 100, and 200 mM DTT, 3.0 mM dNTPs, a reaction buffer [50 mM Tris-HCl, 10 mM (Na₄)₂SO₄, 10 mM MgCl₂, 35 mM DTT, pH 7.4], and 0.75 U/μL phi29 DNA polymerase for 24 h at 26° C.

TABLE 1
DNA sequences for synthesizing circular DNA

| Strand | DNA sequences |
|---|---|
| Linear ssDNA | 5′-Phosphate-TCG TTT GAT |
| (pH-responsive | GTT CCA AAA GAT CGT ATG |
| sequences; | GGT TAG GGT TAG GGT TAG |
| i-motif) | GGA TAC GAT CAA AAC TGA |
| | TGT TGA GGG GGG TCA ACA |
| | TCA GAA AAT GTC TG-3′ |
| Linear ssDNA | 5′-Phosphate-TCA AAC GAC |
| (anti-nucleolin | AGA CAA AAA ACC ACC ACC |
| sequences; | ACC ACA ACC ACC ACC ACC |
| AS1411) | AAA AAA AAC CAC CAC CAC |
| | CAC AAC CAC CAC CAC |
| | CAA AAA GGA ACA-3′ |
| Primer #1 ssDNA | 5′-GGAACATCAAACGACAGACA-3′ |
| Primer #2 ssDNA 5 | 5′-TGTCTGTCGTTTGATGTTCC-3′ |

* Bold letter indicates pH-responsive functional sequences and bold/underline indicates cell adhesion functional sequences.
Normal letter/underline indicates complementary binding region.

(Morphological And Structural Analyses)

A field emission scanning electron microscope (FE-SEM; JSM-7001F; JEOL, Tokyo, Japan) was used to obtain high-resolution digital images of the pDNets for morphological characterization. For SEM observation, pDNets were dried on a silicon wafer and observed at an accelerating voltage of 5 kV. A STEM (JEM-F200; JEOL, Tokyo, Japan) was used for the structural characterization of crystallinity-tuned pDNets at an accelerating voltage of 200 kV. For sample preparation, pDNets were deposited on a Formvar/carbon-coated copper grid (3430C-FA; SPI Supplies, West Chester, PA, USA) and air-dried at room temperature. CLSM (LSM 700; Carl Zeiss, Thornwood, NY, USA) was used to observe the distribution of the polymeric DNA after staining with SYBR Green II (Thermo Fisher Scientific, Waltham, MA, USA). PXRD patterns were collected using a Rigaku Ultima IV diffractometer with Cu Kα radiation at room temperature. The measurements were obtained at 40 kV and 40 mA with a step size of 0.02°. Data points were collected from 5° to 40° at a scan rate of 1°/min.

(Physicochemical Analyses of Crystallinity-Controlled pDNets)

A UV-vis spectrophotometer (V-650; JASCO Corporation, Tokyo, Japan) and RT-PCR thermocycler (CFX96; Bio-Rad Laboratories Inc., Hercules, CA, USA) were used to observe the crystal synthesis and DNA polymerization rates at different DTT: MgCl₂ ratios, respectively. Similar to the synthesis of crystallinity-controlled pDNets, mixtures of circular DNAs at the final concentration of 0.3 μM with 5, 200, and 300 mM DTT, 3.0 mM dNTP, a reaction buffer [50 mM Tris-HCl, 10 mM (Na₄)₂SO₄, 10 mM MgCl₂, 35 mM DTT, pH 7.4], and 0.75 U/μL phi29 DNA polymerase were used for characterization. For UV-Vis analysis, changes in absorbance over time were observed at intervals of 6 min throughout the incubation period of 21 h at 24° C. For RT-PCR analysis, 125×SYBR Green II was added to the RCA solutions and the fluorescence intensities resulting from the intercalation of SYBR Green II were measured at intervals of 10 min throughout the incubation period until the fluorescence intensity was saturated. EDS analysis of the pDNets was performed to compare the atomic compositions with STEM-based EDS mapping at 200 kV. Additionally, the total amount of replicated DNA of each pDNet was investigated after treatment with 500 mM EDTA for 2 h at 40° C. for the decrystallization of MgPPi. The concentration of decrystallized DNA of each type of pDNet was measured using a NanoDrop spectrophotometer (Nanodrop ND-1000; Thermo Fisher Scientific, Waltham, MA, USA).

(Mechanical Characterization of Crystallinity-Controlled pDNets)

The mechanical properties of crystallinity-controlled pDNets were monitored using a rheometer (MCR 102, Anton Paar, Graz, Australia) with parallel plate geometry (diameter of 1.5 mm) at a gap size of 0.25 mm. The storage (G′) and loss (G″) moduli of pDNets were obtained by performing a frequency sweep from 0.1 to 10 Hz at a constant shear strain of 5%. Additionally, an amplitude sweep was performed from a shear strain of 10-100000% with an angular frequency of 1 Hz.

(Drug Loading And Release of Crystallinity-Controlled pDNets)

To monitor the drug loading kinetics, pDNets were incubated in 0.5 mL Dox solution (0.1 mM) for 4 h at room temperature. The change in fluorescence of the Dox supernatant was monitored using a multi-label microplate reader (Victor X5; Perkin Elmer, Waltham, MA, USA), which was used to calculate the drug loading capacity using the following equation:

$$\frac{[ Initial Dox - Free Dox in supernatant ]}{Initial concentration} \times 100$$

The pH-responsive release of the intercalated Dox under different pH conditions was analyzed by incubating pDNets in PBS at pH 5.0, 6.4, and 7.4. Similar to the drug loading profile, the release profile was obtained by measuring the Dox fluorescence of the supernatant using a multi-label microplate reader. Additionally, the enzyme-responsive release profile of Dox was evaluated by treating intercalated Dox with 70, 140, 280, and 560 units/mL DNase I and observing the fluorescence intensity.

(In Vitro Evaluation of Crystallinity-Controlled pDNets)

Cell adhesion efficiencies were evaluated by incubating GelRed-labeled pDNets in cell media containing 2.5×10⁵ Hela or NIH/3T3 cells for an hour. After the incubation, Calcein AM was used to stain the attached cells and the pDNets were washed three times with PBS. After staining, CLSM was used to visualize the respective fluorescence to determine the number of cells attached to each pDNet/mm². Evaluation of intracellular delivery of released Dox from pDNets was performed by loading pDNets with 20 nmol Dox. Dox-loaded pDNets were incubated in a pH 5.0, 6.4, and 7.5 PBS solution for 32 h, which were then diluted 1, 2, and 4-fold with Dulbecco's modified Eagle's medium and used to treat 1×10⁵ HeLa cells for 2 h. As a positive control, 20 nmol free Dox diluted similarly was used for treatment at final concentrations of 2, 4, and 8 μM to represent complete release of Dox. After the incubation, cell nuclei were stained with Hoechst and the intracellular delivery of Dox was compared using CLSM. To verify the cytotoxicity of the released Dox, pDNets were first loaded with 50 nmol Dox and incubated under each pH condition (5.0, 6.4, and 7.4) for 32 h to induce pH-dependent Dox release. The incubation solutions were diluted 100-fold and added to Hela cells seeded in a 96-well plate at the density of 3×10³ cells/well. After 24 h of treatment, cell viability was quantified using the MTT assay (Roche Diagnostics, Mannheim, Germany) following the manufacturer's protocol. In short, the MTT reagent was mixed with the growth medium at a ratio of 1:9 and incubated with networks at 37° C. until formazan salts were formed. The resulting formazan salts were dissolved by adding dimethyl sulfoxide, and the absorbance was measured at 570 nm using a multi-label microplate reader (Victor X5; Perkin Elmer).

(In Vivo Evaluation of Localized Anticancer Therapy)

All animal experiments were performed in compliance with the relevant laws and institutional guidelines of the Korea Institute of Science and Technology (KIST; KIST-2020-108). To evaluate the anticancer effects of pDNets, tumor-bearing BALB/c nude mice were prepared by injecting a suspension of 5×10⁶ cells per mouse. The mice were divided into five groups (n=5): (1) PBS, (2) free DOX, (3) pDNet-H, (4) pDNet-M, and (5) pDNet-L. Each mouse was injected intratumorally with 2.5 mg/kg Dox when the size of the tumors was approximately 100-150 mm ³. The tumor volumes and body weights of each mouse were observed every 2 days for 8 days. The length (L) and width (W) of the tumors were measured, and tumor volumes (V) were calculated using the following formula: V=L×W²×0.52. To assess the biocompatibility of the pDNets, mice in each group were sacrificed after 8 days and the major organs were excised. To assess the biosafety of pDNets, the excised organs were paraffin-embedded and cut into 5 μm sections. Hematoxylin and eosin (H&E) staining was performed to observe the histopathological changes.

(Statistical Analysis)

Experimental data are expressed as mean±standard deviation (SD) for three samples per group. Differences between groups were analyzed using one-way analysis of variance (ANOVA) with the Scheffe test, and the function of the SPSS software package version 24.0. The data were marked as *p<0.05; **p<0.01; ***p<0.005.

### Experimental Example 1 Fabrication of Crystallinity-Controlled and Functional Polymeric DNA Networks

Crystallinity-controlled functional polymeric DNA networks (pDNet) were engineered by designing two types of circular DNAs with functional DNA sequences responsible for: 1) chemodrug loading via intercalation and pH-responsive drug release (e.g., i-motif) and 2) minimizing the undesired side effects via active tumor targeting using a nucleolin-specific aptamer (e.g., AS1411). Also, the circular DNAs were designed to contain a binding region (˜20 bp) complementary to the other regions for structural support (see Table 1). During RCA, the Φ29 DNA polymerase requires Mg for DNA replication, and it produces pyrophosphate (PPi⁻) as a by-product (FIG. 1B).[49] The produced PPi molecule binds with Mg in the buffer to form MgPPi, which induces nucleation of an inorganic crystalline structure that electrostatically complexes with the produced polymeric DNA. As the rate of DNA replication (PPi formation) and concentration of Mg are the key factors that control crystal formation, the ratio of DTT to MgCl₂ was manipulated to influence the rate of crystal growth of the RCA product, as DTT is known as a chelating agent that induces interaction between Mg²⁺ and its hydroxyl groups.[50] Under our optimized conditions, the ratio of DTT and MgCl₂ concentrations were adjusted to 0.5, 10, and 20 for appropriate crystallinity control.

### Experimental Example 2 Microscopic Morphology of Crystallinity-Controlled Polymeric DNA Networks

The fabricated RCA products exhibited different transparencies (FIG. 2A). Replication of DNA was compared by staining pDNets with DNA-intercalating dye (SYBR Green II) and observing the fluorescence with a fluorescence microscope and a confocal laser scanning microscope (CLSM). Green fluorescence, indicative of the presence of polymerized DNA, was observed in all samples (FIG. 2B). Interestingly, the CLSM results also indicated changes in the DNA localization pattern with crystallinity, as the number of microstructures increased with the DTT: MgCl₂ ratio. To further investigate changes in the polymerized DNA localization pattern and composite morphology, the resultant pDNets were analyzed using scanning electron microscopy (SEM). Consistent with the CLSM results, the SEM revealed a reduction in the number of spherulitic porous microstructures of the network and an increase in the amorphous region with the decrease in the DTT: MgCl₂ ratio (FIG. 2C). pDNet synthesized using a DTT: MgCl₂ ratio of 0.5 exhibited the highest number of crystalline microstructures (approximately 1 μm), whereas negligible crystalline structures were observed for pDNet synthesized using a DTT: MgCl₂ ratio of 20, indicating the controllability of crystallinity in pDNets. Therefore, the synthesized pDNets were labeled as high, medium, and low crystallinity pDNets (pDNet-H, pDNet- M, and pDNet-L, respectively), which refer to the degree of crystallinity. Similar to the structural characterization in our previous report, the results of scanning transmission electron microscopy (STEM) of pDNet-H indicated that it consists of hierarchical and multi-layered crystal sheet structures, with strong contrast between the polymeric DNA and MgPPi crystal regions (FIG. 2D).[29] As expected, the STEM results of pDNet-M and pDNet-L indicated a reduction in the crystallinity of pDNet, demonstrated by weak to no contrasting differences between the polymeric DNA and MgPPi crystal regions. Overall, these structural and morphological features of pDNets clearly indicated manipulation of crystallinity and DNA localization patterns upon varying the DTT to MgCl₂ ratio (FIG. 2E).

### Experimental Example 3 Elemental Analyses of Crystallinity-Controlled Polymeric DNA Networks

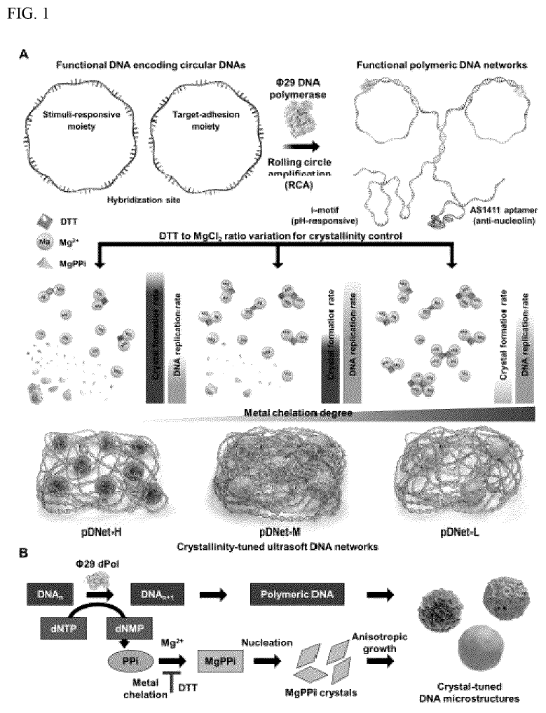

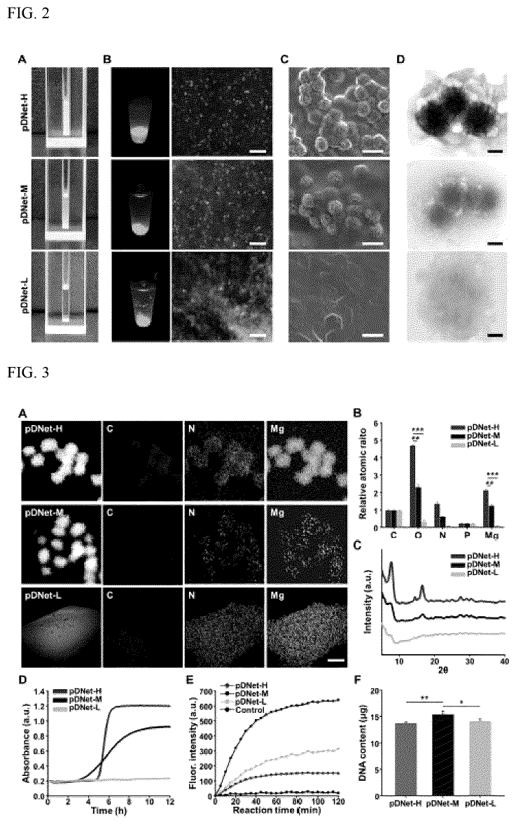

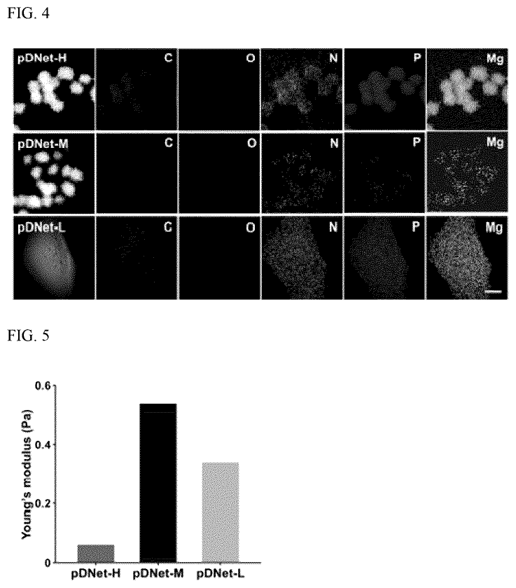

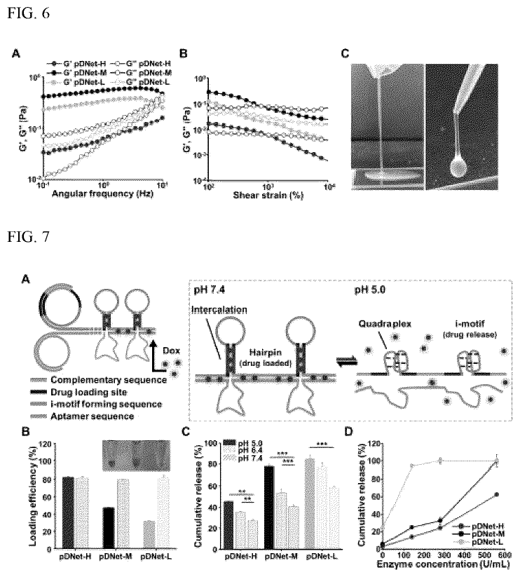

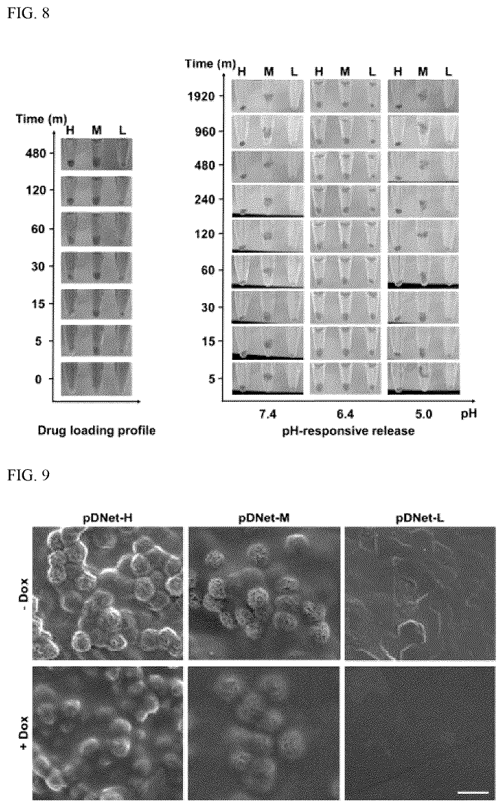

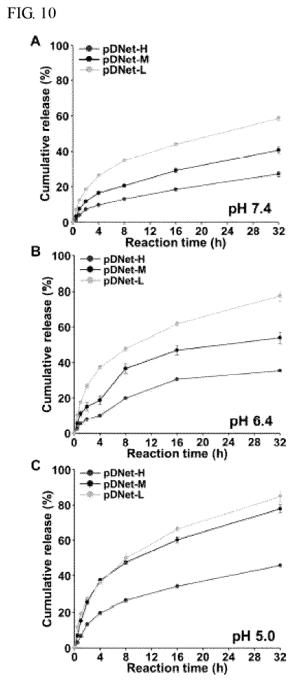

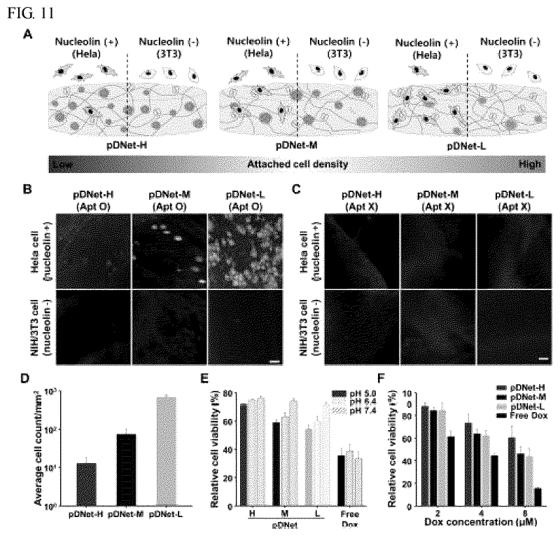

To confirm the distribution of the inorganic MgPPi and organic DNA, elemental analysis was performed using STEM-based energy dispersive spectroscopy (EDS). The results of EDS confirmed the elemental compositions of C, N, O, Mg, and P in the pDNets (FIG. 3A, FIG. 4). High Mg. P, and O atomic weight percentages confirmed the presence of MgPPi in the nanosheets in the core of the particle. Moreover, the presence of C indicates that pDNets include DNA and that they are evenly distributed throughout the surface of the particle. Based on the results of quantitative EDS, the crystal-to-DNA ratios were compared by calculating the atomic ratios relative to C, which is indicative of MgPPi crystal content relative to DNA (FIG. 3B). The relative ratios of Mg and O, indicative of the MgPPi crystal structure, were the lowest in pDNet-L and significantly higher in pDNet-M and pDNet-H, confirming successful manipulation of the crystal-to-DNA ratio by controlling the DTT: MgCl₂ ratio. Additionally, the crystallinity intensities of the pDNets that were synthesized in the same volume were compared using powder X-ray diffraction (PXRD). The diffraction patterns of the pDNets were similar to those of Mg₂P₂O₇·3.5 H₂O in inorganic structural databases and identical to those of the previously reported MgPPi crystal patterns (FIG. 3C). The diffraction intensities were considerably lower for pDNet-L and pDNet-M than for pDNet-H, indicating a reduction in crystallinity. Overall, the morphological and structural characterizations of pDNets confirmed successful control of the MgPPi crystal to polymeric DNA network ratio in a one-pot reaction.

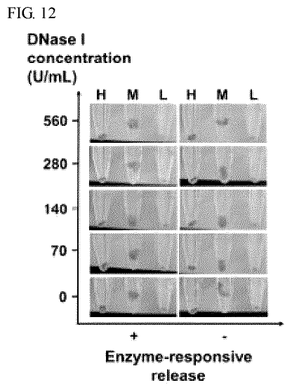

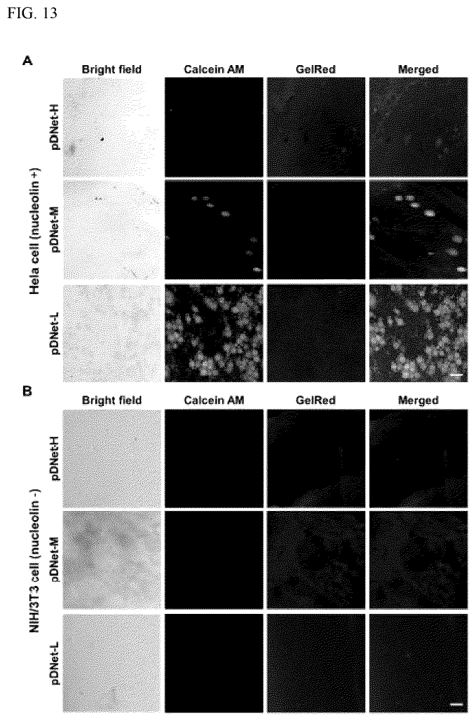

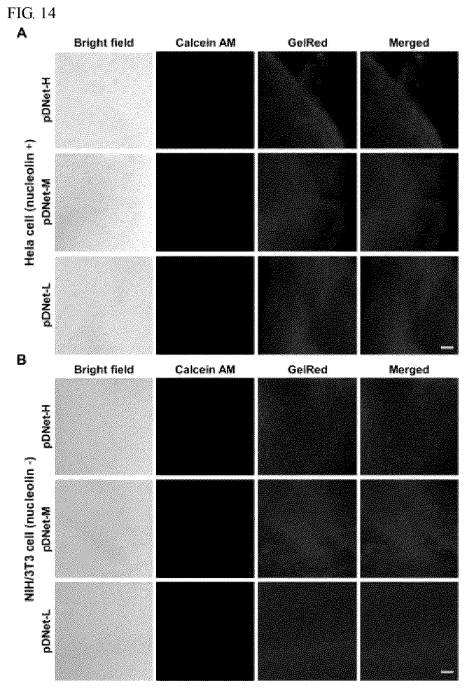

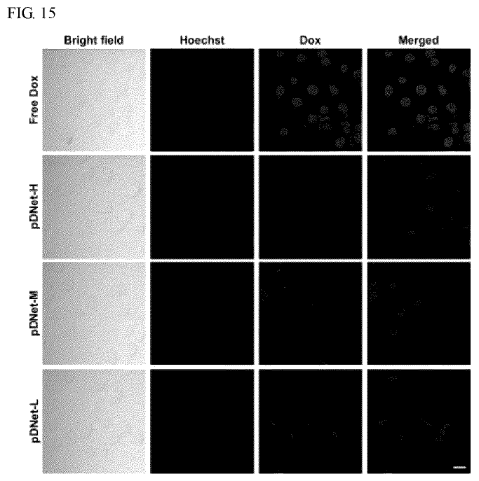

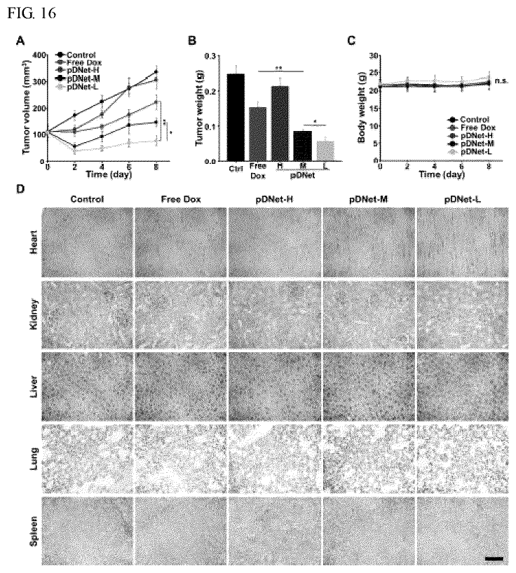

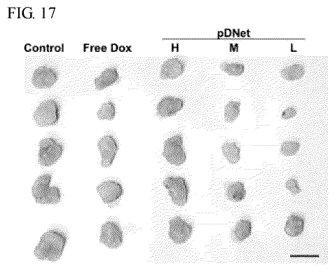

To further investigate the effects of the DTT: MgCl₂ ratio on RCA kinetics, a UV-visible (vis) spectrophotometer and real-time polymerase chain reaction (RT-PCR) were utilized to analyze the growth rate of MgPPi crystals and the amplification rate of the polymerized DNA, respectively. First, the synthesis rate of the crystal structure was confirmed by analyzing the transparency of the pDNets at 520 nm (FIG. 3D). Considering that DNA transmits in the visible region (260 nm), an increase in absorbance at 520 nm indicated the growth of a crystal structure. The highest crystal growth rate was observed in pDNet-H, followed by those in pDNet-M and pDNet-L, and the absorbance saturation levels were dependent on the degree of crystallinity. Similarly, RT-PCR was used to examine the DNA amplification rate by analyzing the changes in fluorescence intensity of SYBR Green II that intercalates with the amplified DNA strands over enzymatic reaction time (FIG. 3E). The DNA amplification rate was also confirmed to be dependent on the degree of crystallinity, possibly owing to the difference in the available Mg ions in the RCA buffer. It was speculated that the differences arise from the DTT: MgCl₂ ratio, as DTT may have interacted with the Mg as a chelating agent, thereby hindering efficient DNA amplification by the Mg-dependent DNA polymerase. The efficiency of DNA production was evaluated by monitoring the total amount of polymeric DNA produced in each pDNet after eliminating MgPPi crystals with EDTA treatment. Batches of 50 mL each of pDNet-H, pDNet-M, and pDNet-L produced 5023, 12259, and 5949 ng polymeric DNA, respectively (FIG. 5).

From the above results, the decrease in DNA production of pDNet-L was attributed to the hindrance to DNA replication due to a reduction in free Mg ion concentration by DTT-mediated chelation, resulting in gradual crystal-free production of polymeric DNA networks with tandem repeats of functional sequences. In the case of pDNet-H, rapid formation of MgPPi crystals from the abundant free Mg ions hindered the diffusion of enzyme reaction substrates (e.g., dNTP). Therefore, it was speculated that the highest production rate of pDNet-M was because of the ability to overcome the conditions posed by the hindering factors described above.

### Experimental Example 4 Rheological Properties of Crystallinity-Controlled Polymeric DNA Networks

The effects of crystal-to-DNA ratio manipulation on the mechanical properties of pDNet were investigated using a rheometer, as the stiffness and elasticity of the network plays an important role in injectability. Crystallinity-controlled pDNets were analyzed using frequency sweep and amplitude sweep analyses. In the frequency sweep analysis, all pDNets exhibited solid-like behavior at a frequency of 1 Hz, as the storage modulus (G′) was larger than the loss modulus (G″; FIG. 6A). Importantly, pDNets were extremely soft, as the Young's moduli were measured to be 0.06, 0.54, and 0.34 Pa for pDNet-H, pDNet-M, and pDNet-L, respectively (FIG. 6B). The mechanical property of pDNets were able to be controlled by approximately 9-fold by varying the degree of crystallinity. Shear-thinning behavior was also observed for pDNet-M and pDNet-L, indicating that its mechanical properties were suitable for injection. It is noteworthy that the network behavior against the shear rate can be tailored simply by controlling the degree of crystallinity for possible bioapplications. Interestingly, pDNet-H was shown to be the softest despite the high content of crystal structure that was expected to hold together polymeric DNA via electrostatic and van der Waals dispersion interactions over a larger surface area. In comparison, the structure of pDNet-L is possibly maintained mainly by physical entanglement of the polymeric DNAs and hydrogen bonds from partial complementary sequence binding, resulting in a stiffer network than that of pDNet-H. pDNet-M exhibited the highest Young's modulus, possibly because of the balance between elastic interactions and hydrogen bonds. However, in the case of amplitude sweep, the critical strain was measured to be 1030%, 775%, and 326% for pDNet-H, pDNet-M, and pDNet-L, respectively, indicating network disruption proportional to the crystallinity (FIG. 6C). Along with the shear-thinning behavior, the high elasticity and liquid-like behavior of pDNet is favorable for injection using a syringe without any post processing steps (e.g., gelation), which is generally required for injectable networks (FIG. 6D). These results confirmed the changes in RCA kinetics and demonstrated the potential of designing fabricated DNA networks with injectable and tunable ultrasoft mechanical properties.

### Experimental Example 5 Responsive Drug Release of Crystallinity-Controlled Polymeric DNA Networks

As minimization of side effects and immune responses are critical for the success of a drug delivery platform, regulation of its disease-targeting property, divalent ion composition, and injectability may be beneficial. Therefore, the applicability of pDNet as a stimuli-responsive drug delivery platform was analyzed by investigating the loading and release profiles of the DNA-intercalating anticancer drug, doxorubicin (Dox), and assessing the preservation of the pH-responsiveness of the i-motif sequences after polymerization. In a circular DNA, the reversible formation of the i-motif structure in acidic tumor environments (pH 5.0 and 6.4) should induce disruption of the hairpin structure that is used as a drug-loading region, inducing pH-responsive release of Dox (FIG. 7A). In the experimental example, the loading and release profiles were controlled by adjusting the crystallinity of the pDNets, which influenced the localization, nanoporosity, and diffusion rate of Dox. The drug loading profile was determined by observing the changes in the fluorescence intensity of Dox in the supernatant during the incubation period (FIG. 8). After 2 h of drug loading, the loading efficiencies of pDNets varied drastically because of differences in surface area, resulting in drug loading efficiencies of 82.4±1.1, 47.6±0.6, and 32.4±0.1% for pDNet-H, pDNet-M, and pDNet-L, respectively (FIG. 7B). However, with an increase in the duration of incubation, the pDNets were able to similarly incorporate 81.6±1.2, 79.4±0.4, and 82.2±1.8% Dox, respectively. In terms of pH-responsive drug release, the pDNets partially released the intercalated Dox when incubated in pH 7.4 PBS, possibly because of the degradation of the DNA network (FIG. 7C), which is consistent with the results of previously reported studies.[52] Based on the cumulative amount of released Dox, it can be concluded that the mechanical stability of pDNet correlated with crystallinity, as Dox was released in the order of pDNet-L, pDNet-M, and pDNet-H (FIG. 9). As expected, upon incubation of pDNets in PBS at pH 5.0, the cumulative release of Dox increased for all samples. Interestingly, as the change in cumulative drug release is because of the i-motif structural switch, the efficiency in sequence-guided functionality was dependent on crystallinity, and pDNet-L exhibited the highest cumulative release, followed by pDNet-M and pDNet-H. These results indicated that the crystal structure of pDNet interferes with the secondary structure formation of functional DNA sequences, suggesting the importance of crystallinity control.

Finally, the enzyme-stability of pDNets was investigated by observing the release of Dox during incubation under harsh DNase I conditions (up to 560 U/mL) for 1 h (FIG. 7D, FIG. 10). All pDNets exhibited gradual release in a DNase concentration-dependent manner, where pDNet-L and pDNet-M were mostly degraded when incubated with 120 and 560 U/mL DNase, respectively. In contrast, despite the soft viscoelastic modulus, pDNet-H exhibited the maximum resistance to enzyme-mediated degradation, as approximately 60% of the intercalated Dox was released when incubated under harsh enzymatic conditions (560 U/mL DNase). Considering that the physiological concentration of DNase I is approximately 0.01 U/mL, the pDNets exhibited extremely high stability. Similar to the crystallinity-dependent sequence function, the difference in stability was attributed to the differences in the microenvironment of pDNets due to the presence of crystals that interfere with enzyme-DNA binding.

### Experimental Example 6 Targeted Cell Adhesion of Crystallinity-Controlled Polymeric DNA Networks

To validate the second sequence-mediated functionality, the aptamer-induced cell adhesion efficiency of pDNets was evaluated by incubating GelRed-labeled pDNets with cell media containing nucleolin-positive HeLa cells or nucleolin-negative NIH/3T3 cells (FIG. 11A). After an hour of incubation, the adhered cells were stained with calcein AM, followed by several washing steps. As illustrated by the green fluorescence in the CLSM images, pDNets exhibited crystallinity-dependent attachment to HeLa cells, the efficiency of which tended to be similar to the i-motif structure formation efficiency (FIG. 11B, FIG. 12). In contrast, pDNets incubated with NIH/3T3 cells, there was no cell attachment, indicating target disease specificity. To confirm that the difference in cell attachment did not result from differences in mechanical property, pDNets with scrambled sequences were incubated with HeLa and NIH/3T3 cells. Similar to the results obtained using NIH/3T3 cells, cell adhesion was not observed for any pDNet, confirming dependency on the functional sequence (FIG. 11C, FIG. 13). The average numbers of Hela cells attached to pDNets per area were quantified using ImageJ and were calculated to be 13±5.5, 76±25.0, and 701±84.1 cells/mm² for pDNet-H pDNet-M, and pDNet-L, respectively (FIG. 11D). These results again highlighted the effectiveness and importance of crystalline structure control in sequence-mediated functionalities, as pDNet-H exhibited the largest surface area available for cell binding, but the lowest cell attachment.

### Experimental Example 7 Drug Delivery Application of Crystallinity-Controlled Polymeric DNA Networks

Finally, the applicability of pDNets as a cancer therapeutic was investigated in vitro and in vivo. Prior to evaluation of the in vitro cancer therapeutic effect, intracellular delivery of Dox released from pDNets in a tumor environment (pH 6.4) was validated using a CLSM. As indicated by red fluorescence, intracellular delivery of Dox was successful for all sample groups, which showed similar drug release profiles (FIG. 14). To investigate the in vitro therapeutic efficacy, pDNets with the same initial concentration were incubated in solutions of different pH (5.0, 6.4, and 7.4). MTT assay of cells treated with each incubation solution indicated the highest therapeutic effect for pDNet-L at pH 5.0, which was consistent with the results of the pH-responsive release experiment (FIG. 11E FIG. 15). The applicability of pDNets for localized cancer therapy was evaluated by investigating whether cell adhesion and drug release efficiencies are maintained in vivo after intratumoral injection of pDNets loaded with 2.5 mg/kg Dox into HeLa cell-implanted BALB/c nude mice xenografts (n=5). The efficacy of controlled release of Dox, evaluated by the decrease in tumor volume during the treatment period, followed the order of pDNet-L>pDNet-M>pDNet-H (FIG. 16A). Eight days after the injection, mice were sacrificed as sample groups exhibited significant tumor volume differences. Tumors of each sample group were harvested for capturing the ex vivo images (FIG. 16B). Quantitatively, relative to the control group, both tumor volume and weight were reduced approximately by 75% for pDNet-L and by 35% for free Dox without pDNets (FIG. 16C). These results revealed the importance of encapsulating therapeutic agents in a drug delivery platform in enhancing anticancer efficacy and disease cell targetability. The difference in the therapeutic efficacy between pDNet-H and pDNet-L was attributed to the differences in structure formation efficiencies of the i-motif and aptamer sequences. Also, formation of accurate 3D arrangements of the incorporated aptamer sequences may have contributed to the adherence of the network to the aptamer-specific tumor region using the ultrasoft mechanical property of the physically entangled DNA networks. More importantly, although free Dox was the most effective in vitro, pDNet-L and -M were more effective in vivo owing to the prolonged tumor residual time and the continuous supplementation of drugs from the implanted network. Consistent with the benefits of the localized drug delivery platform, body weight analyses indicated negligible toxicity in all sample groups (FIG. 16D). Additionally, the biosafety of pDNets was evaluated using hematoxylin-eosin H&E staining of the major organs: heart, kidneys, liver, lungs, and spleen. In conjunction with the results of body weight analyses, tissues from mice of all groups did not exhibit any significant damage, confirming the biosafety of the localized drug delivery platform (FIG. 17). Overall, the crystallinity-dependent drug release, structural stability under physiological conditions, ultrasoft viscoelastic mechanical property, sequence-mediated functionality, and multi-stimuli responsiveness demonstrated that the pDNet system can be tailored for localized cancer therapy and controlled drug delivery in vivo.

What is claimed is:

1. A method for preparing a polymeric DNA network with controlled crystallinity, the method comprising:
hybridizing a first single-stranded circular DNA comprising a stimulus-sensitive moiety and a first hybridization site and a second single-stranded circular DNA comprising a target-attachment moiety and a second hybridization site; and
synthesizing a polymeric DNA network through rolling circle amplification (RCA) in a reaction solution comprising the hybridized circular DNA, DNA polymerase, magnesium chloride (MgCl₂) and dithiothreitol (DTT),
wherein the crystallinity of MgPPi crystals in the polymeric DNA network is controlled by controlling the molar (M) ratio of DTT to magnesium chloride (MgCl₂) from 0.1 to 50.

2. The method of claim 1, wherein as the molar (M) ratio of DTT to magnesium chloride (MgCl₂) decreases, the crystallinity of the MgPPi crystals in the polymeric DNA network increases.

3. The method of claim 1, wherein the stimulus-sensitive moiety is selected from among a pH-sensitive I-motif, a biological material-sensitive aptamer, an ion-sensitive G-quadraplex, a heat-sensitive double helix structure, a base sequence-sensitive toe-hold structure, a base sequence and an element-sensitive DNAzyme.

4. The method of claim 1, wherein the first single-stranded circular DNA comprises a base sequence of SEQ ID NO: 1.

5. The method of claim 1, wherein the target-attachment moiety is selected from among aptamers specific for any one of cell receptors, ATP, ions and metal particles.

6. The method of claim 1, wherein the second single-stranded DNA comprises a base sequence of SEQ ID NO: 2.

7. The method of claim 1, wherein the first single-stranded circular DNA and the second single-stranded circular DNA further comprise a primer.

8. The method of claim 1, wherein the first hybridization site and the second hybridization site comprise 10 to 40 complementary nucleotides.

9. A polymeric DNA network prepared by the method of claim 1.

10. A drug delivery system comprising the polymeric DNA network of claim 9; and a pharmaceutically acceptable carrier.

11. An anticancer composition comprising the polymeric DNA network of claim 9; and an anticancer drug.

12. A method for treating cancer, the method comprising administering a therapeutically effective amount of the anticancer composition of claim 11 to a subject in need thereof.


SEQUENCE LISTING (4 sequences)

SEQ ID NO: 1 - DNA, 95 bp, synthetic construct
  source 1..95: mol_type = other DNA
tcgtttgatgttccaaaagatcgtatgggttagggttagggttagggatacgatcaaaactgatgttgaggggggtcaacatcagaaaatgtctg

SEQ ID NO: 2 - DNA, 90 bp, synthetic construct
  source 1..90: mol_type = other DNA
tcaaacgacagacaaaaaaccaccaccaccacaaccaccaccaccaaaaaaaaccaccaccaccacaaccaccaccaccaaaaaggaaca

SEQ ID NO: 3 - DNA, 20 bp, synthetic construct
  source 1..20: mol_type = other DNA
ggaacatcaaacgacagaca

SEQ ID NO: 4 - DNA, 20 bp, synthetic construct
  source 1..20: mol_type = other DNA
tgtctgtcgtttgatgttcc